(19) United States
(12) Patent Application Publication
(10) Pub. No.: US 2024/0108555 A1
(43) Pub. Date: Apr. 4, 2024

(54) UNREACTIVE TRANSFER SPLINTS

(71) Applicant: VOCO GmbH, Cuxhaven (DE)

(72) Inventors: Sebastian Lamping, Münster (DE); Markus Nordmann, Cuxhaven (DE); Daniel Oldenburger, Cuxhaven (DE); Manfred Thomas Plaumann, Cuxhaven (DE)

(21) Appl. No.: 18/467,465

(22) Filed: Sep. 14, 2023

(30) Foreign Application Priority Data
Sep. 15, 2022 (DE) 10 2022 123 585.8

(51) Int. Cl. A61K 6/887

(52) U.S. Cl. CPC A61K 6/887

(57) ABSTRACT

The present invention relates to free-radical-polymerizable compositions and to the use thereof in stereolithography and/or 3D printing, especially for the 3D printing of dental shaped components, such as dental transfer splints, IBTs, transfer keys and drilling templates. The free-radical-polymerizable compositions have the characteristic feature that, after polymerization, they exhibit practically no adhesion to other (meth)acrylic systems.

### CROSS REFERENCE TO RELATED APPLICATIONS

This application claims priority to German Patent Application No. 102022123585.8, filed Sep. 15, 2022, which is herein incorporated by reference in its entirety.

### FIELD OF THE DISCLOSURE

The present invention relates to a free-radical-polymerizable composition and to the use thereof in stereolithography and/or 3D printing, preferably for the 3D printing of dental shaped components, such as dental transfer splints, IBTs, transfer keys and drilling templates.

### BACKGROUND OF THE DISCLOSURE

With the constant advances in digital dentistry, additive manufacturing processes have in the meantime become established both in dentist practices and in dental laboratories. Data on the individual tooth situation are often obtained with a 3D scanner and can be processed on a computer. On the basis of these data, 3D printing or stereolithography can then be used to print dental shaped components according to the particular application, for example models, trays, dentures, splints, occlusal splints, orthodontic shaped components, indirect bonding trays (IBTs), casts, drilling templates, crowns or bridges.

Additive manufacturing processes, especially stereolithographic processes for producing dental shaped bodies, are known from the prior art.

WO 97/29901 A1 specifies a process for producing a three-dimensional article from a curable liquid medium, where the article is built up layer-by-layer by each time applying a layer of liquid medium to a support and/or an already formed part of the article in a vessel containing liquid medium and subsequently curing said layer.

WO 2013/153183 A2 describes composite resin compositions and processes for producing dental components by stereolithography. The document claims the use of a dental composition comprising a polyreactive binder, two photopolymerization initiators having different absorption maxima and an absorber.

Polymerizable compositions, especially (meth)acrylic compositions, normally have a certain proportion of unreacted double bonds. This has the advantage that (meth)acrylic compositions bond very well with already polymerized (meth)acrylic compositions. This is utilized inter alia in direct filling treatment, where for example a filling material adheres very well to a photopolymerized bonding. Likewise, the basis for the incremental technique is that each increment optimally adheres to a previous, polymerized increment and so gives rise to a homogeneous composite filling.

There are however also applications in which adhesion or bonding are exactly what is not desired. Impressions, transfer keys, transfer splints, IBTs or templates must not form any bond with the material being worked with alongside them. Therefore, if (meth)acrylic systems are being worked with, silicones are commonly employed.

However, there is at the same time also a desire also to produce impressions, transfer keys, transfer splints, IBTs or templates by photopolymerization, through 3D printing based on precise 3D data.

There is thus a demand for free-radical-polymerizable compositions, especially (meth)acrylic compositions that after polymerization exhibit no adhesion or no appreciable adhesion to other (meth)acrylic compositions.

### BRIEF DESCRIPTION OF THE DISCLOSURE

In one aspect, the present disclosure is directed to a dental, free-radical-polymerizable composition comprising
A1) one or more polymerizable (meth)acryloyl compounds of the formula I

Y—O—[(C₃H₆O)ᵥ-L¹]ₙ—(C₃H₆O)_{w}—Z (I)

wherein
Y=H₂C═C(R¹)—C(═O)— or H₂C═C(R¹)—C(═O)—X-L²-,
R¹=H or CH₃,
X=O or NH,
L¹=divalent organic linker group,
L²=divalent organic linker group,
Z═Y, H or C1 to C4 alkyl,
v=36 to 100,
w=36 to 200,
n=0 to 3,
n*v+w=36 to 200
A2) one or more monofunctional (meth)acryloyl compounds that have one (meth)acryloyl group and that do not correspond to formula I,
A3) one or more difunctional (meth)acryloyl compounds that have two (meth)acryloyl groups and that do not correspond to formula I,
B) a photoinitiator or a photoinitiator system.

In one aspect, the composition additionally comprises A4) one or more multifunctional (meth)acryloyl compounds having three to six (meth)acryloyl groups.

In another aspect, the disclosure is directed to dental, free-radical-polymerizable composition as described herein, for use in stereolithography and/or 3D printing, preferably for the 3D printing of dental shaped components, preferably for the 3D printing of dental transfer splints, IBTs, transfer keys and drilling templates.

Other aspects of the disclosure are described in detail hereinafter.

### DETAILED DESCRIPTION OF THE DISCLOSURE

In one aspect, the disclosure is directed to a dental, free-radical-polymerizable composition comprising
A1) one or more polymerizable (meth)acryloyl compounds of the formula I

Y—O—[(C₃H₆O)ᵥ-L¹]ₙ—(C₃H₆O)_{w}—Z (I)
wherein
Y=H₂C═C(R¹)—C(═O)— or H₂C═C(R¹)—C(═O)—X-L²-,
R¹=H or CH₃,
X=O or NH,
L¹=divalent organic linker group,
L²=divalent organic linker group,
Z═Y, H or C1 to C4 alkyl,
v=36 to 100,
w=36 to 200,
n=0 to 3,
n*v+w=36 to 200
A2) one or more monofunctional (meth)acryloyl compounds that have one (meth)acryloyl group and that do not correspond to formula I,
A3) one or more difunctional (meth)acryloyl compounds that have two (meth)acryloyl groups and that do not correspond to formula I, and
B) a photoinitiator or a photoinitiator system.

(Meth)acryloyl compounds are understood here as meaning both acryloyl compounds and methacryloyl compounds.

In a preferred embodiment, a composition according to the invention additionally comprises one or more multifunctional (meth)acryloyl compounds (A4) having three to six (meth)acryloyl groups.

The use of monomers comprising polypropylene groups in 3D printing is known per se. For instance, WO 2019/175716 A1 describes a plurality of corresponding monomers. However, the monomers mentioned therein do not exhibit the desired effect. It is probable that the PPG units are not large enough.

WO 2022/097667 A1 also describes light-curable resin compositions comprising polyalkylene glycol monomers. The compositions are used for producing models in casting processes and are distinguished by low soot generation, which should result in less cracking. Longer-chain monomers are disclosed only in connection with PEG units.

It has surprisingly been found that the combination of a (meth)acryloyl compound (A1) of the formula (I), which has one or more large PPG groups, with the (meth)acryloyl compounds (A2), (A3) and optionally (A4) as well as a photoinitiator (B) leads to compositions that after polymerization exhibit practically no adhesion to other (meth)acrylic systems.

Clearly, a synergistic effect arises here between the PPG domains on the one hand and the combination of the mono-, di- and multifunctional monomers on the other. Whereas the di- and multifunctional monomers form a three-dimensional network around the PPG domains, the mobile monofunctional monomers result in largely full saturation of the residual free double bonds in the network. This gives rise to shaped articles that are essentially unreactive towards further (meth)acrylic monomers.

Polymerizable (meth)acryloyl Compound (A1)

In one aspect, a composition according to the disclosure comprises 30% to 70% by weight, preferably 40% to 60% by weight of polymerizable (meth)acryloyl compounds of the formula I wherein

Y—O—[(C₃H₆O)ᵥ-L¹]ₙ—(C₃H₆O)_{w}—Z (I)
wherein
   Y=H₂C═C(R¹)—C(═O)— or H₂C═C(R¹)—C(═O)—X-L²-,
   R¹=H or CH₃,
   X=O or NH,
   L¹=divalent organic linker group,
   L²=divalent organic linker group,
   Z═Y, H or C1 to C4 alkyl,
   v=36 to 100,
   w=36 to 200,
   v+w=75 to 200 and
   n=0 to 3.

In a preferred embodiment, in the (meth)acryloyl compound of the formula I, X═O, Z═Y, n=0 and w=75 to 180, preferably 75 to 150, more preferably 100 to 150.

In a further preferred embodiment, in the (meth)acryloyl compound of the formula I, X═O, Z═Y, n=1 to 3, v=36 to 100, preferably 36 to 90, more preferably 36 to 80, w=36 to 100, preferably 36 to 90, more preferably 36 to 80, and v+w=75 to 180, preferably 75 to 150, more preferably 100 to 150.

In a further preferred embodiment, in the (meth)acryloyl compound of the formula I, X═O, Z═Y, n=0 to 3, v=36 to 100, preferably 36 to 90, more preferably 36 to 80, w=36 to 100, preferably 36 to 90, more preferably 36 to 80, and n*v+w=75 to 200, preferably 75 to 180, more preferably 100 to 150.

In a preferred embodiment, in the (meth)acryloyl compound of the formula I, L¹ is a straight-chain, branched and/or cyclic alkylene, arylene, arylalkylene or alkylarylene group, which can contain —O—, —OC(═O)—, —C(═O)O—, —NHC(═O)—, —C(═O)NH—, —OC(═O)NH—, or —NHC(═O)O—, and L² is a straight-chain, branched and/or cyclic alkylene, arylene, arylalkylene or alkylarylene group, which can contain —O—, —OC(═O)—, —C(═O)O—, —NHC(═O)—, —C(═O)NH—, —OC(═O)NH—, or —NHC(═O)O—.

In one aspect, preferably, L¹ and/or L² is/are selected from the group consisting of

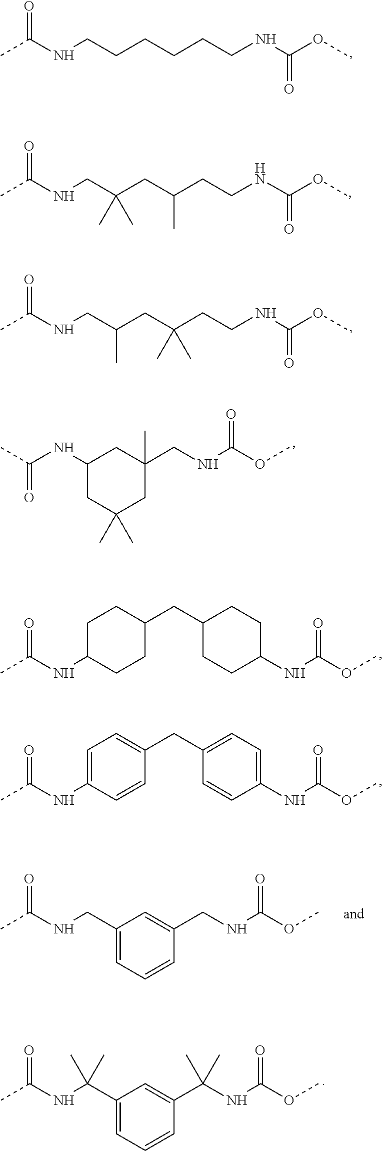

In another aspect, L¹ and/or L² is/are preferably selected from the group consisting of

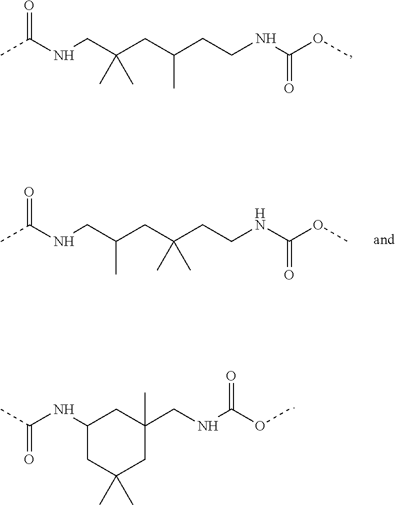

In these compounds, L¹ is attached by the left-hand dashed line to the outer oxygen atom of the polypropylene group (C₃H₆O)ᵥ and is attached by the right-hand dashed line to the outer carbon atom of the polypropylene group (C₃H₆O)_{w}, and/or L² is attached by the left-hand dashed line to X and is attached by the right-hand dashed line, via an oxygen atom (—O—), to the outer carbon atom of one of the polypropylene groups (C₃H₆O)_{w} or (C₃H₆O)ᵥ.

In a preferred embodiment, the (meth)acryloyl compound of the formula I has a molecular weight of greater than 5000 g/mol, preferably of greater than 6000 g/mol, more preferably of greater than 7000 g/mol, and/or in the range from 5000 to 15 000 g/mol, preferably in the range from 6000 to 12,000 g/mol, more preferably in the range from 7000 to 10,000 g/mol.

Monofunctional (meth)acryloyl Compound (A2)

In one aspect, a composition according to the disclosure comprises 10% to 50% by weight, preferably 20% to 50% by weight, of polymerizable monofunctional (meth)acryloyl compounds (A2), based on the total mass of the polymerizable composition.

In a preferred embodiment, the monofunctional (meth)acryloyl compound (A2) is selected from the group consisting of 2-hydroxyethyl (meth)acrylate, 2-hydroxypropyl (meth)acrylate, 3-hydroxypropyl (meth)acrylate, tetrahydrofurfuryl (meth)acrylate, 4-(meth)acryloylmorpholine, cyclohexyl (meth)acrylate, 3,3,5-trimethylcyclohexyl (meth)acrylate, 4-(1,1-dimethylethyl)cyclohexyl (meth)acrylate, isobornyl (meth)acrylate, (octahydro-4,7-methano-1H-indenyl)methyl (meth)acrylate, benzyl (meth)acrylate, 2-phenoxyethyl (meth)acrylate, 2-(2-phenoxyethoxy)ethyl (meth)acrylate, 2-[2-(2-phenoxyethoxy)ethoxy]ethyl (meth)acrylate, ethoxylated 2-phenoxyethyl(meth)acrylate 2-ethoxyethyl (meth)acrylate, 2-(2-ethoxyethoxy)ethyl (meth)acrylate, 2-[2-(2-ethoxyethoxy)ethoxy]ethyl (meth)acrylate, 2-methoxy ethyl (meth)acrylate, 2-(2-methoxy ethoxy)ethyl (meth)acrylate, 2-[2-(2-methoxyethoxy)ethoxy]ethyl (meth)acrylate, hexyl (meth)acrylate, 2-ethylhexyl (meth)acrylate, decyl (meth)acrylate, isodecyl (meth)acrylate, dodecyl (meth)acrylate, 2-hydroxy-3-(prop-2-enoyloxy)propyl 2-methyl-2-propylhexanoate and (5-ethyl-1,3-dioxan-5-yl)methyl (meth)acrylate.

Difunctional (meth)acryloyl Compound (A3)

In one aspect, a composition according to the disclosure comprises 1% to 30% by weight, preferably 2% to 15% by weight, of polymerizable difunctional (meth)acryloyl compounds (A3), based on the total mass of the polymerizable composition.

In a preferred embodiment, the difunctional (meth)acryloyl compound (A3) is selected from the group consisting of ethylene glycol di(meth)acrylate, diethylene glycol di(meth)acrylate, triethylene glycol di(meth)acrylate, tetraethylene glycol di(meth)acrylate, polyethylene glycol di(meth)acrylate, propylene glycol di(meth)acrylate, dipropylene glycol di(meth)acrylate, tripropylene glycol di(meth)acrylate, tetrapropylene glycol di(meth)acrylate, polypropylene glycol di(meth)acrylate, butane-1,4-diol di(meth)acrylate, butane-1,3-diol di(meth)acrylate, hexane-1,6-diol di(meth)acrylate, decane-1,10-diol di(meth)acrylate, dodecane-1,12-diol di(meth)acrylate, 2-hydroxypropyl 1,3-di(meth)acrylate, 3-hydroxypropyl 1,2-di(meth)acrylate, neopentyl glycol di(meth)acrylate, 2,2-bis[4-[3-(meth)acryloyloxy-2-hydroxypropoxy]phenyl]propane, 2,2-bis[4-(meth)acryloyloxyphenyl]propane, 2,2-bis[4-(meth)acryloyloxyethoxyphenyl]propane, 2,2-bis[4-(meth)acryloyloxydiethoxyphenyl]propane, 2,2-bis[4-(meth)acryloyloxytriethoxyphenyl]propane 2,2-bis[4-(meth)acryloyloxytetraethoxyphenyl]propane, 2,2-bis[4-(meth)acryloyloxypentaethoxyphenyl]propane, 2,2-bis[4-(meth)acryloyloxydipropoxyphenyl]propane, 2,2-bis[4-(meth)acryloyloxyethoxyphenyl]-2-[4-(meth)acryloyloxydiethoxyphenyl]propane, 2-[4-(meth)acryloyloxydiethoxyphenyl]-2-[4-(meth)acryloyloxytriethoxyphenyl]propane, 2-[4-(meth)acryloyloxydipropoxyphenyl]-2-[4-(meth)acryloyloxytriethoxyphenyl]propane, 2,2-bis[4-(meth)acryloyloxyisopropoxyphenyl]propane, ethoxylated bisphenol A di(meth)acrylate, propoxylated bisphenol A di(meth)acrylate, 3(4),8(9)-bis((meth)acryloyloxymethyl)tricyclo[5.2.1.0^{2,6}]decane, 3,14-dioxa-4,13-dioxo-5,12-diazahexadecane-1,16-diyl di(meth)acrylate, 7,7,9-trimethyl-3,14-dioxa-4,13-dioxo-5,12-diazahexadecane-1,16-diyl di(meth)acrylate, 7,9,9-trimethyl-3,14-dioxa-4,13-dioxo-5,12-diazahexadecane-1,16-diyl di(meth)acrylate, 1,5,5-trimethyl-1-[(2-(meth)acryloyloxyethyl)carbamoylmethyl]-3-(2-(meth)acryloyloxyethyl)carbamoylcyclohexane (CAS 42405-01-6 methacrylate; CAS 42404-50-2 acrylate), 1,1′-[methylenebis(4,1-phenylenimino-carbonyloxy-2,1-ethanediyl)] bis(meth)acrylate (CAS 51243-61-9 methacrylate; CAS 69790-08-5 acrylate) and 1,1′-[methylenebis(2,1-phenyleniminocarbonyloxy-2,1-ethanediyl)] bis(meth)acrylate.

Multifunctional (meth)acryloyl Compound (A4)

In one aspect, a composition according to the disclosure comprises 1% to 20% by weight, preferably 1% to 10% by weight, of polymerizable multifunctional (meth)acryloyl compounds (A4), based on the total mass of the polymerizable composition.

In a preferred embodiment, the multifunctional (meth)acryloyl compound (A4) is selected from the group consisting of glycerol tri(meth)acrylate, ethoxylated glycerol tri(meth)acrylate, propoxylated glycerol tri(meth)acrylate, trimethylolethane tri(meth)acrylate, ethoxylated trimethylolethane tri(meth)acrylate, trimethylolpropane tri(meth)acrylate, ethoxylated trimethylolpropane tri(meth)acrylate, ditrimethylolpropane tri(meth)acrylate, ditrimethylolpropane tetra(meth)acrylate, pentaerythritol tri(meth)acrylate, pentaerythritol tetra(meth)acrylate, ethoxylated pentaerythritol tetra(meth)acrylate, dipentaerythritol tri(meth)acrylate, dipentaerythritol tetra(meth)acrylate, dipentaerythritol penta(meth)acrylate, di pentaerythritol hexa(meth)acrylate, ethoxylated dipentaerythritol penta(meth)acrylate, ethoxylated dipentaerythritol hexa(meth)acrylate and (2,4,6-trioxo-1,3,5-triazine-1,3,5(2H,4H,6H)-triyl)tri-2,1-ethanediyl tri(meth)acrylate.

### Photoinitiators (B)

In one aspect, a composition according to the disclosure comprises 0.1% to 5% by weight, preferably 0.5% to 4% by weight, in each case based on the total mass of the polymerizable composition, of a photoinitiator or photoinitiator system (B) for the free-radical polymerization. Photoinitiators that may be used are those compounds suitable and customary for (meth)acrylic systems. Advantageously used are alpha-diketones, benzoin alkyl ethers, thioxanthones, benzophenones, acetophenones, acylphosphine oxides or acylgermanium compounds. Preference is given to using monoacylphosphine oxides or bisacylphosphine oxides.

In a preferred configuration, (B) is selected from the group consisting of alpha-diketones, benzoin alkyl ethers, thioxanthones, benzophenones, acetophenones, acylphosphine oxides and acylgermanium compounds, preferably selected from the group consisting of 1-hydroxycyclohexylbenzophenone, 4-(2-hydroxyethoxy)phenyl 2-hydroxy-2-propyl ketone, 2-hydroxy-2-methylpropiophenone, 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate (CAS 84434-11-7) and (poly(oxy-1,2-ethanediyl),α,α′,α″-1,2,3-propanetriyltris[ω-[[phenyl(2,4,6-trimethylbenzoyl)phosphinyl]oxy] polymer) (CAS 1834525-17-5), more preferably selected from the group consisting of 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate and (poly(oxy-1,2-ethanediyl),α,α′,α″-1,2,3-propanetriyltris[ω-[[phenyl(2,4,6-trimethylbenzoyl)phosphinyl]oxy] polymer).

### Stabilizers/Inhibitors (C)

In a preferred embodiment, a composition according to the disclosure comprises 0.001% to 1% by weight, preferably 0.01% to 0.5% by weight, more preferably 0.01% to 0.3% by weight, in each case based on the total mass of the polymerizable composition, of one or more stabilizers (C).

Preferably, (C) is selected from the group consisting of hydroquinone, hydroquinone monomethyl ether, 2,6-di-tert-butyl-4-methylphenol, tert-butylhydroxyanisole and 2,2,6,6-tetramethylpiperidine-1-oxyl.

In a preferred embodiment, a composition according to the disclosure comprises:
A1) in an amount of from 30% to 70% by weight, preferably 40% to 60% by weight,
A2) in an amount of from 10% to 50% by weight, preferably 20% to 50% by weight,
A3) in an amount of from 1% to 30% by weight, preferably 2% to 15% by weight,
B) in an amount of from 0.1% to 5% by weight, preferably 0.5% to 4% by weight,
in each case based on the total mass of the polymerizable composition.

In a further preferred embodiment, a composition according to the disclosure comprises:
A1) in an amount of from 30% to 70% by weight, preferably 40% to 60% by weight,
A2) in an amount of from 10% to 50% by weight, preferably 20% to 50% by weight,
A3) in an amount of from 1% to 30% by weight, preferably 2% to 15% by weight,
A4) in an amount of from 1% to 20% by weight, preferably 1% to 10% by weight,
B) in an amount of from 0.1% to 5% by weight, preferably 0.5% to 4% by
in each case based on the total mass of the polymerizable composition.

In a further preferred embodiment, a composition according to the disclosure comprises:
A1) in an amount of from 30% to 70% by weight, preferably 40% to 60% by weight,
A2) in an amount of from 10% to 50% by weight, preferably 20% to 50% by weight,
A3) in an amount of from 1% to 30% by weight, preferably 2% to 15% by weight,
A4) in an amount of from 1% to 20% by weight, preferably 1% to 10% by weight,
B) in an amount of from 0.1% to 5% by weight, preferably 0.5% to 4% by weight, and
C) in an amount of from 0.001% to 1% by weight, preferably 0.01% to 0.5% by weight,
in each case based on the total mass of the polymerizable composition.

Preference is also given to a composition according to the disclosure in which (meth)acryloyl compound (A1) contains at least one urethane group and/or (preferably and) (meth)acryloyl compound (A2) does not contain a urethane group and/or (preferably and) (meth)acryloyl compound (A3) does not contain a urethane group and/or (preferably and) (meth)acryloyl compound (A4) does not contain a urethane group.

As described above, the combination of monomers (A1), (A2), (A3) and optionally (A4) results in a printing resin that after polymerization exhibits no appreciable adhesion to other (meth)acrylic systems.

Also in accordance with the invention is a dental, free-radical-polymerizable composition as described above, for use in stereolithography and/or 3D printing, preferably for the 3D printing of dental shaped components, preferably for the 3D printing of dental transfer splints, IBTs, transfer keys and drilling templates.

Also in accordance with the invention is the use of a dental, free-radical-polymerizable composition as described above in stereolithography and/or 3D printing, preferably for the 3D printing of dental shaped components, preferably for the 3D printing of dental transfer splints, IBTs, transfer keys and drilling templates.

Also in accordance with the invention is a dental, free-radical-polymerizable composition as described above, for use in a procedure for the therapeutic or surgical treatment of the human body, preferably for specific use in a therapeutic procedure for correcting misaligned teeth or in a surgical procedure for drilling implant holes.

### Embodiments

The invention can be summarized overall on the basis of the aspects that follow.
1. Dental, free-radical-polymerizable composition comprising
A1) one or more polymerizable (meth)acryloyl compounds of the formula I

Y—O—[(C₃H₆O)ᵥ-L¹]ₙ—(C₃H₆O)_{w}—Z (I)

where
Y=H₂C═C(R′)—C(═O)— or H₂C═C(R′)—C(═O)—X-L²-,
R¹=H or CH₃,
X=O or NH,
L¹=divalent organic linker group,
L²=divalent organic linker group,
Z═Y, H or C1 to C4 alkyl,
v=36 to 100,
w=36 to 200,
n=0 to 3,
n*v+w=36 to 200
A2) one or more monofunctional (meth)acryloyl compounds that have one (meth)acryloyl group and that do not correspond to formula I,
A3) one or more difunctional (meth)acryloyl compounds that have two (meth)acryloyl groups and that do not correspond to formula I,
B) a photoinitiator or a photoinitiator system.
2. Dental, free-radical-polymerizable composition according to aspect 1, additionally comprising
A4) one or more multifunctional (meth)acryloyl compounds having three to six (meth)acryloyl groups.
3. Dental, free-radical-polymerizable composition according to aspect 1, comprising (A1) in an amount of from 30% to 70% by weight, preferably 40% to 60% by weight, based on the total mass of the polymerizable composition.
4. Dental, free-radical-polymerizable composition according to any of the preceding aspects, wherein, in the (meth)acryloyl compound of the formula I, X=O, Z═Y, n=0 and w=75 to 180, preferably 75 to 150, more preferably 100 to 150.
5. Dental, free-radical-polymerizable composition according to any of the preceding aspects, wherein, in the (meth)acryloyl compound of the formula I, X=O, Z═Y, n=1 to 3, v=36 to 100, preferably 36 to 90, more preferably 36 to 80, w=36 to 100, preferably 36 to 90, more preferably 36 to 80, and v+w=75 to 180, preferably 75 to 150, more preferably 100 to 150.
6. Dental, free-radical-polymerizable composition according to any of the preceding aspects, wherein, in the (meth)acryloyl compound of the formula I, X=O, Z═Y, n=0 to 3, v=36 to 100, preferably 36 to 90, more preferably 36 to 80, w=36 to 100, preferably 36 to 90, more preferably 36 to 80, and n*v+w=75 to 200, preferably 75 to 180, more preferably 100 to 150.
7. Dental, free-radical-polymerizable composition according to any of the preceding aspects, wherein, in the (meth)acryloyl compound of the formula I,
L¹ is a straight-chain, branched and/or cyclic alkylene, arylene, arylalkylene or alkylarylene group, which can contain —O—, —OC(═O)—, —C(═O)O—, —NHC(═O)—, —C(═O)NH—, —OC(═O)NH—, or —NHC(═O)O—, and
L² is a straight-chain, branched and/or cyclic alkylene, arylene, arylalkylene or alkylarylene group, which can contain —O—, —OC(═O)—, —C(═O)O—, —NHC(═O)—, —C(═O)NH—, —OC(═O)NH—, or —NHC(═O)O—.
8. Dental, free-radical-polymerizable composition according to any of the preceding aspects, wherein L¹ and/or L² is/are selected from the group consisting of

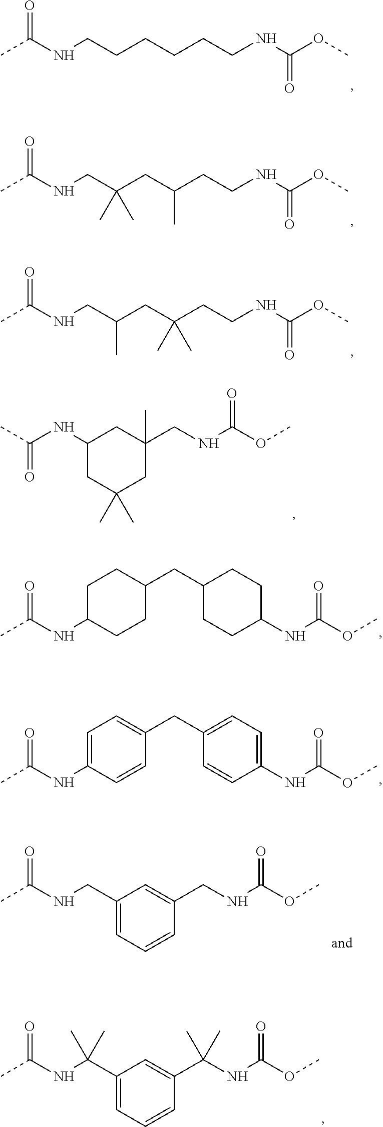

and preferably selected from the group consisting of

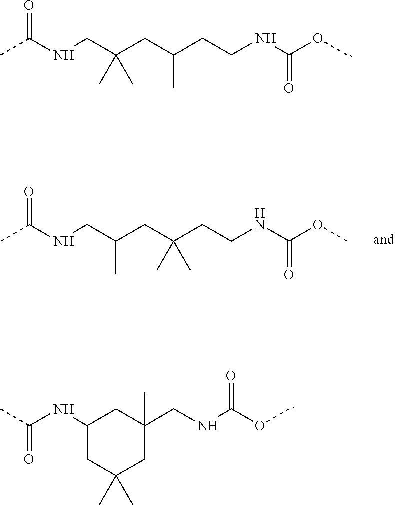

9. Dental, free-radical-polymerizable composition according to any of the preceding aspects, wherein L¹ and/or L² is/are selected from the group consisting of

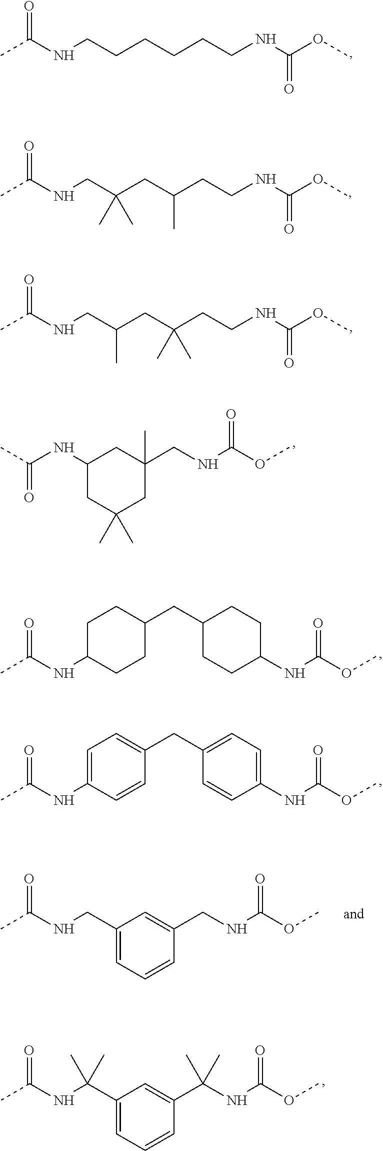

and preferably selected from the group consisting of

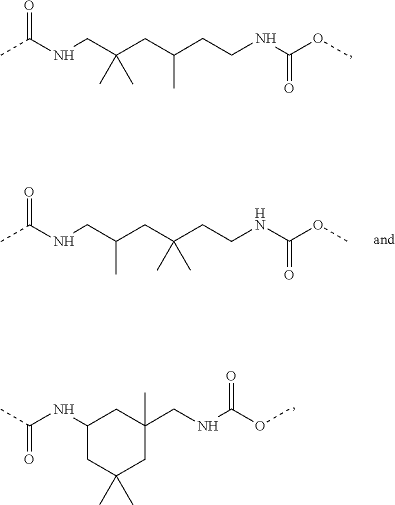

where L¹ is attached by the left-hand dashed line to the outer oxygen atom of the polypropylene group (C₃H₆O), and is attached by the right-hand dashed line to the outer carbon atom of the polypropylene group (C₃H₆O)_{w}, and/or L² is attached by the left-hand dashed line to X and is attached by the right-hand dashed line, via an oxygen atom (—O—), to the outer carbon atom of one of the polypropylene groups (C₃H₆O)_{w} or (C₃H₆O)ᵥ.
10. Dental, free-radical-polymerizable composition according to any of the preceding aspects, wherein the (meth)acryloyl compound of the formula I has a molecular weight of greater than 5000 g/mol, preferably of greater than 6000 g/mol, more preferably of greater than 7000 g/mol, and/or in the range from 5000 to 15 000 g/mol, preferably in the range from 6000 to 12 000 g/mol, more preferably in the range from 7000 to 10 000 g/mol.
11. Dental, free-radical-polymerizable composition according to any of the preceding aspects, comprising (A2) in an amount of from 10% to 50% by weight, preferably 20% to 50% by weight, based on the total mass of the polymerizable composition.
12. Dental, free-radical-polymerizable composition according to any of the preceding aspects, wherein the monofunctional (meth)acryloyl compound (A2) is selected from the group consisting of 2-hydroxyethyl (meth)acrylate, 2-hydroxypropyl (meth)acrylate, 3-hydroxypropyl (meth)acrylate, tetrahydrofurfuryl (meth)acrylate, 4-(meth)acryloylmorpholine, cyclohexyl (meth)acrylate, 3,3,5-trimethylcyclohexyl (meth)acrylate, 4-(1,1-dimethylethyl)cyclohexyl (meth)acrylate, isobornyl (meth)acrylate, (octahydro-4,7-methano-1H-indenyl)methyl (meth)acrylate, benzyl (meth)acrylate, 2-phenoxyethyl (meth)acrylate, 2-(2-phenoxyethoxy)ethyl (meth)acrylate, 2-[2-(2-phenoxyethoxy)ethoxy]ethyl (meth)acrylate, ethoxylated 2-phenoxyethyl(meth)acrylate 2-ethoxyethyl (meth)acrylate, 2-(2-ethoxyethoxy)ethyl (meth)acrylate, 2-[2-(2-ethoxyethoxy)ethoxy]ethyl (meth)acrylate, 2-methoxy ethyl (meth)acrylate, 2-(2-methoxy ethoxy)ethyl (meth)acrylate, 2-[2-(2-methoxyethoxy)ethoxy]ethyl (meth)acrylate, hexyl (meth)acrylate, 2-ethylhexyl (meth)acrylate, decyl (meth)acrylate, isodecyl (meth)acrylate, dodecyl (meth)acrylate, 2-hydroxy-3-(prop-2-enoyloxy)propyl 2-methyl-2-propylhexanoate and (5-ethyl-1,3-dioxan-5-yl)methyl (meth)acrylate.
13. Dental, free-radical-polymerizable composition according to any of the preceding aspects, comprising (A3) in an amount of from 1% to 30% by weight, preferably 2% to 15% by weight, based on the total mass of the polymerizable composition.
14. Dental, free-radical-polymerizable composition according to any of the preceding aspects, wherein the difunctional (meth)acryloyl compound (A3) is selected from the group consisting of ethylene glycol di(meth)acrylate, diethylene glycol di(meth)acrylate, triethylene glycol di(meth)acrylate, tetraethylene glycol di(meth)acrylate, polyethylene glycol di(meth)acrylate, propylene glycol di(meth)acrylate, dipropylene glycol di(meth)acrylate, tripropylene glycol di(meth)acrylate, tetrapropylene glycol di(meth)acrylate, polypropylene glycol di(meth)acrylate, butane-1,4-diol di(meth)acrylate, butane-1,3-diol di(meth)acrylate, hexane-1,6-diol di(meth)acrylate, decane-1,10-diol di(meth)acrylate, dodecane-1,12-diol di(meth)acrylate, 2-hydroxypropyl 1,3-di(meth)acrylate, 3-hydroxypropyl 1,2-di(meth)acrylate, neopentyl glycol di(meth)acrylate, 2,2-bis[4-[3-(meth)acryloyloxy-2-hydroxypropoxy]phenyl]propane, 2,2-bis[4-(meth)acryloyloxyphenyl]propane, 2,2-bis[4-(meth)acryloyloxyethoxyphenyl]propane, 2,2-bis[4-(meth)acryloyloxydiethoxyphenyl]propane, 2,2-bis[4-(meth)acryloyloxytriethoxyphenyl]propane 2,2-bis[4-(meth)acryloyloxytetraethoxyphenyl]propane, 2,2-bis[4-(meth)acryloyloxy pentaethoxy phenyl]propane, 2,2-bis[4-(meth)acryloyloxydipropoxyphenyl]propane, 2,2-bis[4-(meth)acryloyloxy ethoxy phenyl]-2-[4-(meth)acryloyloxydiethoxyphenyl]propane, 2-[4-(meth)acryloyloxydiethoxyphenyl]-2-[4-(meth)acryloyloxytriethoxyphenyl]propane, 2-[4-(meth)acryloyloxydipropoxyphenyl]-2-[4-(meth)acryloyloxytriethoxyphenyl]propane, 2,2-bis[4-(meth)acryloyloxyisopropoxyphenyl]propane, ethoxylated bisphenol A di(meth)acrylate, propoxylated bisphenol A di(meth)acrylate, 3(4),8(9)-bis((meth)acryloyloxymethyl)tricyclo[5.2.1.0^{2,6}]decane, 3,14-dioxa-4,13-dioxo-5,12-diazahexadecane-1,16-diyl di(meth)acrylate, 7,7,9-trimethyl-3,14-dioxa-4,13-dioxo-5,12-diazahexadecane-1,16-diyl di(meth)acrylate, 7,9,9-trimethyl-3,14-dioxa-4,13-dioxo-5,12-diazahexadecane-1,16-diyl di(meth)acrylate, 1,5,5-trimethyl-1-[(2-(meth)acryloyloxyethyl)carbamoylmethyl]-3-(2-(meth)acryloyloxyethyl)carbamoylcyclohexane (CAS 42405-01-6 methacrylate; CAS 42404-50-2 acrylate), 1,1′-[methylenebis(4,1-phenylenimino-carbonyloxy-2,1-ethanediyl)] bis(meth)acrylate (CAS 51243-61-9 methacrylate; CAS 69790-08-5 acrylate) and 1,1′-[methylenebis(2,1-phenyleniminocarbonyloxy-2,1-ethanediyl)] bis(meth)acrylate.
15. Dental, free-radical-polymerizable composition according to any of the preceding aspects, wherein the difunctional (meth)acryloyl compound (A3) is selected from the group consisting of ethylene glycol di(meth)acrylate, diethylene glycol di(meth)acrylate, triethylene glycol di(meth)acrylate, tetraethylene glycol di(meth)acrylate, polyethylene glycol di(meth)acrylate, propylene glycol di(meth)acrylate, dipropylene glycol di(meth)acrylate, tripropylene glycol di(meth)acrylate, tetrapropylene glycol di(meth)acrylate, polypropylene glycol di(meth)acrylate, butane-1,4-diol di(meth)acrylate, butane-1,3-diol di(meth)acrylate, hexane-1,6-diol di(meth)acrylate, decane-1,10-diol di(meth)acrylate, dodecane-1,12-diol di(meth)acrylate, 2-hydroxypropyl 1,3-di(meth)acrylate, 3-hydroxypropyl 1,2-di(meth)acrylate, neopentyl glycol di(meth)acrylate, 2,2-bis[4-[3-(meth)acryloyloxy-2-hydroxypropoxy]phenyl]propane, 2,2-bis[4-(meth)acryloyloxyphenyl]propane, 2,2-bis[4-(meth)acryloyloxyethoxyphenyl]propane, 2,2-bis[4-(meth)acryloyloxydiethoxyphenyl]propane, 2,2-bis[4-(meth)acryloyloxytriethoxyphenyl]propane 2,2-bis[4-(meth)acryloyloxytetraethoxyphenyl]propane, 2,2-bis[4-(meth)acryloyloxy pentaethoxy phenyl]propane, 2,2-bis[4-(meth)acryloyloxydipropoxyphenyl]propane, 2,2-bis[4-(meth)acryloyloxy ethoxy phenyl]-2-[4-(meth)acryloyloxydiethoxyphenyl]propane, 2-[4-(meth)acryloyloxydiethoxyphenyl]-2-[4-(meth)acryloyloxytriethoxyphenyl]propane, 2-[4-(meth)acryloyloxydipropoxyphenyl]-2-[4-(meth)acryloyloxytriethoxyphenyl]propane, 2,2-bis[4-(meth)acryloyloxyisopropoxyphenyl]propane, ethoxylated bisphenol A di(meth)acrylate, propoxylated bisphenol A di(meth)acrylate and 3(4),8(9)-bis((meth)acryloyloxymethyl)tricyclo[5.2.1.0^{2,6}]decane.
16. Dental, free-radical-polymerizable composition according to any of the preceding aspects, comprising (A4) in an amount of from 1% to 20% by weight, preferably 1% to 10% by weight, based on the total mass of the polymerizable composition.
17. Dental, free-radical-polymerizable composition according to any of the preceding aspects, wherein the multifunctional (meth)acryloyl compound (A4) is selected from the group consisting of glycerol tri(meth)acrylate, ethoxylated glycerol tri(meth)acrylate, propoxylated glycerol tri(meth)acrylate, trimethylolethane tri(meth)acrylate, ethoxylated trimethylolethane tri(meth)acrylate, trimethylolpropane tri(meth)acrylate, ethoxylated trimethylolpropane tri(meth)acrylate, ditrimethylolpropane tri(meth)acrylate, ditrimethylolpropane tetra(meth)acrylate, pentaerythritol tri(meth)acrylate, pentaerythritol tetra(meth)acrylate, ethoxylated pentaerythritol tetra(meth)acrylate, dipentaerythritol tri(meth)acrylate, dipentaerythritol tetra(meth)acrylate, dipentaerythritol penta(meth)acrylate, dipentaerythritol hexa(meth)acrylate, ethoxylated dipentaerythritol penta(meth)acrylate, ethoxylated dipentaerythritol hexa(meth)acrylate and (2,4,6-trioxo-1,3,5-triazine-1,3,5(2H,4H,6H)-triyl)tri-2,1-ethanediyl tri(meth)acrylate.
18. Dental, free-radical-polymerizable composition according to any of the preceding aspects, comprising a photoinitiator or photoinitiator system (B) in an amount of from 0.1% to 5% by weight, preferably 0.5% to 4% by weight, in each case based on the total mass of the polymerizable composition.
19. Dental, free-radical-polymerizable composition according to any of the preceding aspects, wherein (B) is selected from the group consisting of alpha-diketones, benzoin alkyl ethers, thioxanthones, benzophenones, acetophenones, acylphosphine oxides and acylgermanium compounds.
20. Dental, free-radical-polymerizable composition according to any of the preceding aspects, wherein (B) is selected from the group consisting of 1-hydroxycyclohexylbenzophenone, 4-(2-hydroxy ethoxy)phenyl 2-hydroxy-2-propyl ketone, 2-hydroxy-2-methylpropiophenone, 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate (CAS 84434-11-7) and (poly(oxy-1,2-ethanediyl), α,α′,α″-1,2,3-propanetriyltris[ω-[[phenyl(2,4,6-trimethylbenzoyl)phosphinyl]oxy] polymer) (CAS 1834525-17-5), more preferably selected from the group consisting of 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate and (poly(oxy-1,2-ethanediyl),α,α′,α″-1,2,3-propanetriyltris[ω-[[phenyl(2,4,6-trimethylbenzoyl)phosphinyl] oxy] polymer).
21. Dental, free-radical-polymerizable composition according to any of the preceding aspects, comprising one or more stabilizers (C) in an amount of from 0.001% to 1% by weight, preferably 0.01% to 0.5% by weight, more preferably 0.01% to 0.3% by weight, in each case based on the total mass of the polymerizable composition.
22. Dental, free-radical-polymerizable composition according to any of the preceding aspects, wherein the stabilizers (C) are selected from the group consisting of hydroquinone, hydroquinone monomethyl ether, 2,6-di-tert-butyl-4-methylphenol, tert-butylhydroxyanisole and 2,2,6,6-tetramethylpiperidine-1-oxyl.
23. Dental, free-radical-polymerizable composition according to any of the preceding aspects, comprising
A1) in an amount of from 30% to 70% by weight, preferably 40% to 60% by weight,
A2) in an amount of from 10% to 50% by weight, preferably 20% to 50% by weight,
A3) in an amount of from 1% to 30% by weight, preferably 2% to 15% by weight,
B) in an amount of from 0.1% to 5% by weight, preferably 0.5% to 4% by weight,
in each case based on the total mass of the polymerizable composition.
24. Dental, free-radical-polymerizable composition according to any of the preceding aspects, comprising
A1) in an amount of from 30% to 70% by weight, preferably 40% to 60% by weight,
A2) in an amount of from 10% to 50% by weight, preferably 20% to 50% by weight,
A3) in an amount of from 1% to 30% by weight, preferably 2% to 15% by weight,
A4) in an amount of from 1% to 20% by weight, preferably 1% to 10% by weight,
B) in an amount of from 0.1% to 5% by weight, preferably 0.5% to 4% by weight,
in each case based on the total mass of the polymerizable composition.
25. Dental, free-radical-polymerizable composition according to any of the preceding aspects, comprising
A1) in an amount of from 30% to 70% by weight, preferably 40% to 60% by weight,
A2) in an amount of from 10% to 50% by weight, preferably 20% to 50% by weight,
A3) in an amount of from 1% to 30% by weight, preferably 2% to 15% by weight,
A4) in an amount of from 1% to 20% by weight, preferably 1% to 10% by weight,
B) in an amount of from 0.1% to 5% by weight, preferably 0.5% to 4% by weight, and
C) in an amount of from 0.001% to 1% by weight, preferably 0.01% to 0.5% by weight,
in each case based on the total mass of the polymerizable composition.
26. Dental, free-radical-polymerizable composition according to any of the preceding aspects, wherein
(meth)acryloyl compound (A1) contains at least one urethane group, and/or (preferably and)
(meth)acryloyl compound (A2) does not contain a urethane group, and/or (preferably and)
(meth)acryloyl compound (A3) does not contain a urethane group, and/or (preferably and)
(meth)acryloyl compound (A4) does not contain a urethane group.
27. Dental, free-radical-polymerizable composition according to any of the preceding aspects, for use in stereolithography and/or 3D printing, preferably for the 3D printing of dental shaped components, preferably for the 3D printing of dental transfer splints, IBTs, transfer keys and drilling templates.
28. Dental, free-radical-polymerizable composition according to any of the preceding aspects, for use in a procedure for the therapeutic or surgical treatment of the human body, preferably for specific use in a therapeutic procedure for correcting misaligned teeth or in a surgical procedure for drilling implant holes.
29. Use of a dental, free-radical-polymerizable composition according to any of the preceding aspects in stereolithography and/or 3D printing, preferably for the 3D printing of dental shaped components, preferably for the 3D printing of dental transfer splints, IBTs, transfer keys and drilling templates.

The invention is defined in the appended claims. Preferred aspects of the present invention emerge moreover from the description herein, including the examples.

Where particular configurations defined for one aspect of the invention (composition or use) are described as preferred, the corresponding statements in each case apply mutatis mutandis for the other aspects of the present invention too. Preferred individual features of aspects of the invention (as defined in the claims and/or disclosed in the description) are combinable with one another and are preferably combined with one another unless in the individual case the present text indicates otherwise to the person skilled in the art.

### EXAMPLES

The aims and advantages of the present disclosure are further illustrated by the examples that follow, however the specific materials and the amounts thereof that are mentioned in these examples, as well as other conditions and details, are not to be interpreted such as to disproportionately limit the present disclosure.

The materials used in the examples are summarized below.
PPG: Polypropylene glycol (CAS 25322-69-4)
PEG: Polyethylene glycol (CAS 25322-68-3)
HMDI: Hexamethylene diisocyanate (CAS 822-06-0)
TMDI: 2,2,4-Trimethylhexamethylene diisocyanate/2,4,4-trimethylhexamethylene diisocyanate (isomer mixture; CAS 32052-51-0)
IPDI: Isophorone diisocyanate (CAS 4098-71-9)
DBTL: Dibutyltin dilaurate (CAS 77-58-7)
BHT: 2,6-Di-tert-butyl-4-methylphenol (CAS 128-37-0)
IBOMA: Isobornyl methacrylate (CAS 7534-94-3)
ACMO: Acryloylmorpholine (CAS 5117-12-4)
THFMA: Tetrahydrofurfuryl methacrylate (CAS 2455-24-5)
PheMA: 2-Phenoxyethyl methacrylate (CAS 10595-06-9)
HEMA: 2-Hydroxyethyl methacrylate (CAS 868-77-9)
CTFA: (5-Ethyl-1,3-dioxan-5-yl)methyl acrylate (CAS 66492-51-1)
BisEMA-6: Ethoxylated bisphenol A dimethacrylate (6 EO units; CAS 41637-38-1)
TCDDMA: 3(4),8(9)-Bis(methacryloyloxymethyl)tricyclo[5.2.1.0^{2,6}]decane (CAS 43048-08-4)
HDDMA: Hexane-1,6-diol dimethacrylate (CAS 6606-59-3)
UDMA: 7,7,9-Trimethyl-3,14-dioxa-4,13-dioxo-5,12-diazahexadecane-1,16-diyl di(meth)acrylate/7,9,9-trimethyl-3,14-dioxa-4,13-dioxo-5,12-diazahexadecane-1,16-diyl di(meth)acrylate (isomer mixture; CAS 72869-86-4)
PETA: Pentaerythritol tetraacrylate (CAS 4986-89-4)
PETA-4: Ethoxylated pentaerythritol tetraacrylate (4 EO units; CAS 51728-26-8)
TMPTA: Trimethylolpropane triacrylate (CAS 15625-89-5)
TMPTA-3: Ethoxylated trimethylolpropane triacrylate (3 EO units; CAS 28961-43-5)
ISCTA: (2,4,6-Trioxo-1,3,5-triazine-1,3,5(2H,4H,6H)-triyl)tri-2,1-ethanediyl triacrylate (CAS 40220-08-4)
TPO: Diphenyl(2,4,6-trimethylbenzoyl)phosphine oxide (CAS 75980-60-8)

### Synthesis of the Polypropylene Glycol (Meth)Acryloyl Compounds

### Example 1a (PPG 5000 dimethacrylate)

100.00 g of PPG 5000 and 4.05 g of triethylamine are dissolved in 400 ml of chloroform. 4.18 g of methacryloyl chloride is dissolved in 20 ml of chloroform and slowly added dropwise at 0° C. At the end of the addition, stirring is continued at 0° C. for one hour. The mixture is then slowly warmed to room temperature and stirred at room temperature for a further 18 hours. The precipitate formed is filtered off and the filtrate is washed twice with water, three times with 10% hydrochloric acid, and twice with saturated sodium chloride solution. The organic phase is dried over magnesium sulfate. 50 mg of BHT is added and the solvent is removed under reduced pressure. A pale yellow liquid is obtained.

### Example 1b (PPG 6000 dimethacrylate)

120.00 g of PPG 6000 and 4.05 g of triethylamine are dissolved in 400 ml of chloroform. 4.18 g of methacryloyl chloride is dissolved in 25 ml of chloroform and slowly added dropwise at 0° C. At the end of the addition, stirring is continued at 0° C. for one hour. The mixture is then slowly warmed to room temperature and stirred at room temperature for a further 18 hours. The precipitate formed is filtered off and the filtrate is washed twice with water, three times with 10% hydrochloric acid, and twice with saturated sodium chloride solution. The organic phase is dried over magnesium sulfate. 50 mg of BHT is added and the solvent is removed under reduced pressure. A pale yellow liquid is obtained.

### Example 1c (PPG 5000 urethane dimethacrylate)

200.00 g of PPG 5000 is dissolved in 400 ml of chloroform. 6.21 g of 2-isocyanatoethyl methacrylate, 200 mg of dibutyltin dilaurate and 100 mg of BHT are then added. The mixture is heated to 55° C. with stirring for 6 hours. The reaction is monitored by observing the disappearance of the NCO band at approx. 2270 cm⁻¹ in the IR spectrum. The solvent is then removed under reduced pressure. A pale yellow liquid is obtained. Viscosity (25° C.): 0.5 Pa*s

### Example 1d (PPG 6000 urethane dimethacrylate)

240.00 g of PPG 6000 is dissolved in 400 ml of chloroform. 6.21 g of 2-isocyanatoethyl methacrylate, 200 mg of dibutyltin dilaurate and 100 mg of BHT are then added. The mixture is heated to 55° C. with stirring for 6 hours. The reaction is monitored by observing the disappearance of the NCO band at approx. 2270 cm⁻¹ in the IR spectrum. The solvent is then removed under reduced pressure. A pale yellow liquid is obtained. Viscosity (25° C.): 0.5 Pa*s

### Example 1e (PPG 5000-HMDI-HEMA)

100.00 g of PPG 5000, 6.73 g of hexamethylene diisocyanate (HMDI) and 5.21 g of 2-hydroxyethyl methacrylate (HEMA) are dissolved in 200 ml of chloroform. 100 mg of dibutyltin dilaurate and 50 mg of BHT are then added. The mixture is heated to 55° C. with stirring for 6 hours. The reaction is monitored by observing the disappearance of the NCO band at approx. 2270 cm⁻¹ in the IR spectrum. The solvent is then removed under reduced pressure. A pale yellow liquid is obtained.

### Example 1f (PPG 5000-TMDI-HEA)

100.00 g of PPG 5000, 8.41 g of trimethylhexamethylene diisocyanate (TMDI) and 4.64 g of 2-hydroxyethyl acrylate (HEA) are dissolved in 200 ml of chloroform. 100 mg of dibutyltin dilaurate and 50 mg of BHT are then added. The mixture is heated to 55° C. with stirring for 6 hours. The reaction is monitored by observing the disappearance of the NCO band at approx. 2270 cm⁻¹ in the IR spectrum. The solvent is then removed under reduced pressure. A pale yellow liquid is obtained.

### Example 1g (PPG 5000-IPDI-HEMA)

100.00 g of PPG 5000, 8.89 g of isophorone diisocyanate (IPDI) and 5.21 g of 2-hydroxyethyl methacrylate (HEMA) are dissolved in 200 ml of chloroform. 100 mg of dibutyltin dilaurate and 50 mg of BHT are then added. The mixture is heated to 55° C. with stirring for 6 hours. The reaction is monitored by observing the disappearance of the NCO band at approx. 2270 cm⁻¹ in the IR spectrum. The solvent is then removed under reduced pressure. A pale yellow liquid is obtained.

### Example 1h (PPG 5000-IPDI-HEA)

100.00 g of PPG 5000, 8.89 g of isophorone diisocyanate (IPDI) and 4.64 g of 2-hydroxyethyl acrylate (HEA) are dissolved in 200 ml of chloroform. 100 mg of dibutyltin dilaurate and 50 mg of BHT are then added. The mixture is heated to 55° C. with stirring for 6 hours. The reaction is monitored by observing the disappearance of the NCO band at approx. 2270 cm⁻¹ in the IR spectrum. The solvent is then removed under reduced pressure. A pale yellow liquid is obtained.

### Example 1i (oligo-PPG 5000-IPDI-HEMA)

100.00 g of PPG 3000, 11.12 g of isophorone diisocyanate (IPDI) and 4.38 g of 2-hydroxyethyl methacrylate (HEMA) are dissolved in 200 ml of chloroform. 100 mg of dibutyltin dilaurate and 50 mg of BHT are then added. The mixture is heated to 55° C. with stirring for 6 hours. The reaction is monitored by observing the disappearance of the NCO band at approx. 2270 cm⁻¹ in the IR spectrum. The solvent is then removed under reduced pressure. A pale yellow liquid is obtained.

### Comparative Example V1a (PPG 2000 dimethacrylate)

40.00 g of PPG 2000 and 4.05 g of triethylamine are dissolved in 200 ml of chloroform. 4.18 g of methacryloyl chloride is dissolved in 25 ml of chloroform and slowly added dropwise at 0° C. At the end of the addition, stirring is continued at 0° C. for one hour. The mixture is then slowly warmed to room temperature and stirred at room temperature for a further 18 hours. The precipitate formed is filtered off and the filtrate is washed twice with water, three times with 10% hydrochloric acid, and twice with saturated sodium chloride solution. The organic phase is dried over magnesium sulfate. 50 mg of BHT is added and the solvent is removed under reduced pressure. A pale yellow liquid is obtained.

### Comparative example V1b (PPG 1000 urethane dimethacrylate)

80.00 g of PPG 1000 is dissolved in 200 ml of chloroform. 12.42 g of 2-isocyanatoethyl methacrylate, 200 mg of dibutyltin dilaurate and 100 mg of BHT are then added. The mixture is heated to 55° C. with stirring for 6 hours. The reaction is monitored by observing the disappearance of the NCO band at approx. 2270 cm⁻¹ in the IR spectrum. The solvent is then removed under reduced pressure. A pale yellow liquid is obtained. Viscosity (25° C.): 1.3 Pa*s

### Comparative example V1c (PPG 4000 urethane dimethacrylate)

160.00 g of PPG 4000 is dissolved in 400 ml of chloroform. 12.42 g of 2-isocyanatoethyl methacrylate, 200 mg of dibutyltin dilaurate and 100 mg of BHT are then added. The mixture is heated to 55° C. with stirring for 6 hours. The reaction is monitored by observing the disappearance of the NCO band at approx. 2270 cm⁻¹ in the IR spectrum. The solvent is then removed under reduced pressure. A pale yellow liquid is obtained. Viscosity (25° C.): 0.5 Pa*s

### Comparative example V1d (PPG 1000-TMDI-HEMA)

40.00 g of PPG 1000, 16.82 g of trimethylhexamethylene diisocyanate (TMDI) and 9.29 g of 2-hydroxyethyl acrylate (HEA) are dissolved in 200 ml of chloroform. 100 mg of dibutyltin dilaurate and 50 mg of BHT are then added. The mixture is heated to 55° C. with stirring for 6 hours. The reaction is monitored by observing the disappearance of the NCO band at approx. 2270 cm⁻¹ in the IR spectrum. The solvent is then removed under reduced pressure. A pale yellow liquid is obtained.

### Example V1e (PEG 6000 dimethacrylate)

120.00 g of PPG 6000 and 4.05 g of triethylamine are dissolved in 400 ml of chloroform. 4.18 g of methacryloyl chloride is dissolved in 25 ml of chloroform and slowly added dropwise at 0° C. At the end of the addition, stirring is continued at 0° C. for one hour. The mixture is then slowly warmed to room temperature and stirred at room temperature for a further 18 hours. The precipitate formed is filtered off and the filtrate is washed twice with water, three times with 10% hydrochloric acid, and twice with saturated sodium chloride solution. The organic phase is dried over magnesium sulfate. 50 mg of BHT is added and the solvent is removed under reduced pressure. A pale yellow liquid is obtained.

### Measurement Methods:

Flexural strength (FS): The flexural strengths were determined in a manner adapted to ISO 4049:2009. Test specimens having dimensions of 40 mm×5 mm×5 mm were produced from the printing resins by 3D printing (SolFlex350, W2P Engineering GmbH; wavelength 385 nm, power 8.3 mW/cm², pixel size 50 μm, layer thickness 50 μm) and were post-exposed using an Otoflash (VOCO GmbH) with 2×2000 flashes. The flexural strength is determined at a forward speed of 1 mm/min on a Zwick universal tester (Zwick GmbH & Co. KG, Ulm).

Adhesion: For the determination of adhesion, test specimens having a diameter of 20 mm and a height of 10 mm were produced from the printing resins by 3D printing (SolFlex350, W2P Engineering GmbH; wavelength 385 nm, power 8.3 mW/cm², pixel size 50 μm, layer thickness 50 μm) and were post-exposed using an Otoflash (VOCO GmbH) with 2×2000 flashes. The surface was cleaned with isopropanol. A silicone ring (internal diameter 5 mm, height 5 mm) is then placed thereon and filled with Structur 2 SC (VOCO GmbH). This is cured for 24 hours at 37° C. and 100% humidity. The silicone ring is then removed and a subjective test of adhesion first takes place. Test specimens for which no bond has formed between the printed test specimen and Structur 2 SC are rated “no adhesion” and the adhesion value is set at 0 MPa. In the case of test specimens for which a bond has formed between the printed test specimen and Structur 2 SC, the adhesion value is determined on a universal tester (Zwick GmbH & Co. KG, Ulm) by loading at a forward speed of 1 mm/min until fracture. The adhesion value is calculated as the ratio of the breaking force (in N) and the adhesion area (in mm 2), in each case as the average value of six individual measurements.

Viscosity: Routine measurements were carried out on an Anton Paar Physica MCR 301 rheometer with a 50 mm measurement plate (plate/plate), 0.5 mm plate distance and 1 g of substance. Prior to the measurement, the plate is thermally equilibrated at a temperature of 25° C. The measurement time is 30 s, at a shear rate of 10/s.

Molecular weight: The molecular weight was determined by GPC (Agilent Technologies 1260 Infinity). The system consists of a guard column (length 50 mm, diameter 8 mm, particle size 5 μm) and two main columns (I: length 300 mm, diameter 8 mm, particle size 5 μm, 50 A; II: length 300 mm, diameter 8 mm, particle size 5 μm, 100 A). The eluent used is THF. The standard used is polystyrene.

### Exemplary Embodiments

The constituents listed in Tables 1 to 6 were each weighed out in the stated amounts and stirred at room temperature with a precision glass stirrer for 60 minutes until a homogeneous solution had formed.

TABLE 1

| Example | | 2a | 2b | 2c | 2d | 2e | 2f |
|---|---|---|---|---|---|---|---|
| (A1) | Monomer 1a | 50.00 | 50.00 | 50.00 | 50.00 | 50.00 | 50.00 |
| (A2) | IBOMA | 30.00 | — | — | — | — | — |
| | ACMO | — | 30.00 | — | — | — | — |
| | THEMA | — | — | 30.00 | — | — | — |
| | PheMA | — | — | — | 30.00 | — | — |
| | HEMA | — | — | — | — | 30.00 | — |
| | CTFA | — | — | — | — | — | 30.00 |
| (A3) | BisEMA-6 | 10.00 | 10.00 | 10.00 | 10.00 | 10.00 | 10.00 |
| (A4) | PETA | 8.50 | 8.50 | 8.50 | 8.50 | 8.50 | 8.50 |
| (B) | TPO | 1.50 | 1.50 | 1.50 | 1.50 | 1.50 | 1.50 |

| | | | | | | |
|---|---|---|---|---|---|---|
| Adhesion [MPa] | 0* | 0* | 0* | 0* | 0* | 0* |

*no bond had formed between the printed test specimen and Structure 2 SC, consequently measurement of adhesion values was not possible.

TABLE 2

| Example | | 2g | 2h | 2i | 2j | 2k | 2l |
|---|---|---|---|---|---|---|---|
| (A1) | Monomer 1a | 50.00 | 50.00 | 50.00 | 50.00 | 55.00 | 45.00 |
| (A2) | IBOMA | 30.00 | 30.00 | 30.00 | 30.00 | 20.00 | 40.00 |
| (A3) | BisEMA-6 | 10.00 | — | — | | 15.00 | 5.00 |
| | TCDDMA | — | 10.00 | — | | — | — |
| | HDDMA | — | — | 10.00 | — | — | — |
| | UDMA | — | — | — | 10.00 | — | — |
| (A4) | PETA | 8.50 | 8.50 | 8.50 | 8.50 | 8.50 | 8.50 |
| (B) | TPO | 1.50 | 1.50 | 1.50 | 1.50 | 1.50 | 1.50 |

| | | | | | | |
|---|---|---|---|---|---|---|
| Adhesion [MPa] | 0* | 0* | 0* | 0* | 0* | 3.3** |

*no bond had formed between the printed test specimen and Structure 2 SC, consequently measurement of adhesion values was not possible.
**in 4 out of 6 test specimens no bond had formed.

TABLE 3

| Example | | 2m | 2n | 2o | 2p | 2q | 2r |
|---|---|---|---|---|---|---|---|
| (A1) | Monomer 1a | 50.00 | 50.00 | 50.00 | 50.00 | 50.00 | 50.00 |
| (A2) | IBOMA | 30.00 | 30.00 | 30.00 | 30.00 | 30.00 | 30.00 |
| (A3) | BisEMA-6 | 10.00 | 10.00 | 10.00 | 10.00 | 15.00 | 8.50 |
| (A4) | PETA | — | — | — | — | 3.50 | 10.00 |
| | PETA-4 | 8.50 | — | — | — | — | — |
| | TMPTA | — | 8.50 | — | — | — | — |
| | TMPTA-3 | — | — | 8.50 | — | — | — |
| | ISCTA | — | — | — | 8.50 | — | — |
| (B) | TPO | 1.50 | 1.50 | 1.50 | 1.50 | 1.50 | 1.50 |

| | | | | | | |
|---|---|---|---|---|---|---|
| Adhesion [MPa] | 0* | 0* | 0* | 0* | 0* | 0* |

*no bond had formed between the printed test specimen and Structure 2 SC, consequently measurement of adhesion values was not possible.

TABLE 4

| Example | | 2s | 2t | 2u | 2v | 2w | 2x |
|---|---|---|---|---|---|---|---|
| (A1) | Monomer 1b | 50.00 | — | — | — | — | — |
| | Monomer 1c | — | 50.00 | — | — | — | — |
| | Monomer 1d | — | — | 50.00 | — | — | — |
| | Monomer 1e | — | — | — | 50.00 | — | — |
| | Monomer 1f | — | — | — | — | 50.00 | — |
| | Monomer 1g | — | — | — | — | — | 50.00 |
| (A2) | IBOMA | 30.00 | 30.00 | 30.00 | 30.00 | 30.00 | 30.00 |
| (A3) | BisEMA-6 | 10.00 | 10.00 | 10.00 | 10.00 | 10.00 | 10.00 |
| (A4) | PETA | 8.50 | 8.50 | 8.50 | 8.50 | 8.50 | 8.50 |
| (B) | TPO | 1.50 | 1.50 | 1.50 | 1.50 | 1.50 | 1.50 |

| | | | | | | |
|---|---|---|---|---|---|---|
| Adhesion [MPa] | 0* | 0* | 0* | 0* | 0* | 0* |

*no bond had formed between the printed test specimen and Structur 2 SC, consequently measurement of adhesion values was not possible.

TABLE 5

| Comparative | | | | | | | |
|---|---|---|---|---|---|---|---|
| example | | 3a | 3b | 3c | 3d | 3e | 3f |
| (A1) | Monomer 1a | 50.00 | 50.00 | 50.00 | — | 10.00 | 80.00 |
| (A2) | IBOMA | — | 40.00 | 30.00 | 60.00 | 55.00 | 15.00 |
| (A3) | BisEMA-6 | 40.00 | — | 18.50 | 20.00 | 18.50 | 2.00 |
| (A4) | PETA | 8.50 | 8.50 | — | 18.50 | 15.00 | 1.50 |
| (B) | TPO | 1.50 | 1.50 | 1.50 | 1.50 | 1.50 | 1.50 |

| | | | | | | |
|---|---|---|---|---|---|---|
| Adhesion [MPa] | 35.7 | 21.8 | 37.4 | 33.1 | 19.4 | 29.7 |

TABLE 6

| Comparative example | | 3g | 3h | 3i | 3j | 3k |
|---|---|---|---|---|---|---|
| | Monomer V1a | 50.00 | — | — | — | — |
| | Monomer V1b | — | 50.00 | — | — | — |
| | Monomer V1c | — | — | 50.00 | — | — |
| | Monomer V1d | — | — | — | 50.00 | — |
| | Monomer V1e | — | — | — | — | 50.00 |
| (A2) | IBOMA | 30.00 | 30.00 | 30.00 | 30.00 | 30.00 |
| (A3) | BisEMA-6 | 10.00 | 10.00 | 10.00 | 10.00 | 10.00 |
| (A4) | PETA | 8.50 | 8.50 | 8.50 | 8.50 | 8.50 |
| (B) | TPO | 1.50 | 1.50 | 1.50 | 1.50 | 1.50 |

| | | | | | |
|---|---|---|---|---|---|
| Adhesion [MPa] | 25.4 | 20.3 | 12.3 | 14.6 | 29.1 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A dental, free-radical-polymerizable composition comprising
A1) one or more polymerizable (meth)acryloyl compounds of the formula I
Y—O—[(C₃H₆O)ᵥ-L¹]ₙ—(C₃H₆O)_{w}—Z (I)
wherein
Y=H₂C═C(R¹)—C(═O)— or H₂C═C(R¹)—C(═O)—X-L²-,
R¹=H or CH 3,
X=O or NH,
L¹=divalent organic linker group,
L²=divalent organic linker group,
Z═Y, H or C1 to C4 alkyl,
v=36 to 100,
w=36 to 200,
n=0 to 3,
n*v+w=36 to 200
A2) one or more monofunctional (meth)acryloyl compounds that have one (meth)acryloyl group and that do not correspond to formula I,
A3) one or more difunctional (meth)acryloyl compounds that have two (meth)acryloyl groups and that do not correspond to formula I and
B) a photoinitiator or a photoinitiator system.

2. The dental, free-radical-polymerizable composition of claim 1, additionally comprising
A4) one or more multifunctional (meth)acryloyl compounds (A4) having three to six (meth)acryloyl groups.

3. The dental, free-radical-polymerizable composition of claim 1, comprising
A1) in an amount of from 30% to 70% by weight,
A2) in an amount of from 10% to 50% by weight,
A3) in an amount of from 1% to 30% by weight,
B) in an amount of from 0.1% to 5% by weight,
in each case based on the total mass of the polymerizable composition.

4. The dental, free-radical-polymerizable composition of claim 2, comprising
A1) in an amount of from 30% to 70% by weight,
A2) in an amount of from 10% to 50% by weight,
A3) in an amount of from 1% to 30% by weight,
A4) in an amount of from 1% to 20% by weight,
B) in an amount of from 0.1% to 5% by weight,
in each case based on the total mass of the polymerizable composition.

5. The dental, free-radical-polymerizable composition of claim 1, wherein, in the (meth)acryloyl compound of the formula I,
X=O,
Z═Y,
n=0 and
w=36 to 200.

6. The dental, free-radical-polymerizable composition of claim 1, wherein, in the (meth)acryloyl compound of the formula I,
X=O,
Z═Y,
n=1 to 3,
v=36 to 100,
w=36 to 100, and
v+w=75 to 180.

7. The dental, free-radical-polymerizable composition of claim 1, wherein, in the (meth)acryloyl compound of the formula I,
L¹ is a straight-chain, branched and/or cyclic alkylene, arylene, arylalkylene or alkylarylene group, which comprises —O—, —OC(═O)—, —C(═O)O—, —NHC(═O)—, —C(═O)NH—, —OC(═O)NH—, or —NHC(═O)O—, and
L² is a straight-chain, branched and/or cyclic alkylene, arylene, arylalkylene or alkylarylene group, which comprises —O—, —OC(═O)—, —C(═O)O—, —NHC(═O)—, —C(═O)NH—, —OC(═O)NH—, or —NHC(═O)O—.

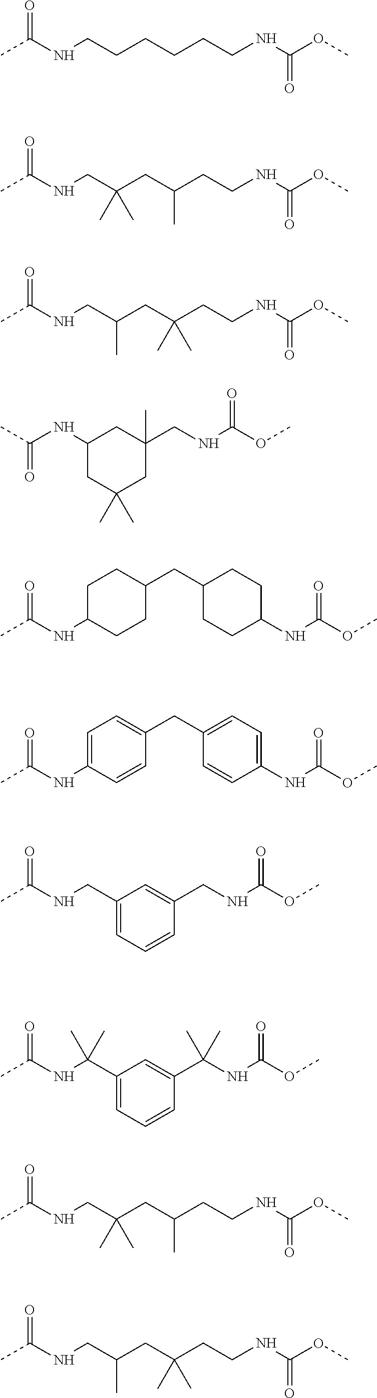

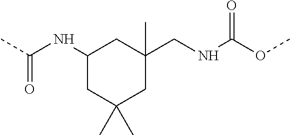

8. The dental, free-radical-polymerizable composition of claim 1, wherein the (meth)acryloyl compound of the formula I has a molecular weight of greater than 5000 g/mol.

9. The dental, free-radical-polymerizable composition of claim 1, wherein the monofunctional (meth)acryloyl compound (A2) is selected from the group consisting of 2-hydroxyethyl (meth)acrylate, 2-hydroxypropyl (meth)acrylate, 3-hydroxypropyl (meth)acrylate, tetrahydrofurfuryl (meth)acrylate, 4-(meth)acryloylmorpholine, cyclohexyl (meth)acrylate, 3,3,5-trimethylcyclohexyl (meth)acrylate, 4-(1,1-dimethylethyl)cyclohexyl (meth)acrylate, isobornyl (meth)acrylate, (octahydro-4,7-methano-1H-indenyl)methyl (meth)acrylate, benzyl (meth)acrylate, 2-phenoxyethyl (meth)acrylate, 2-(2-phenoxyethoxy)ethyl (meth)acrylate, 2-[2-(2-phenoxyethoxy)ethoxy]ethyl (meth)acrylate, ethoxylated 2-phenoxy ethyl (meth)acrylate 2-ethoxyethyl (meth)acrylate, 2-(2-ethoxyethoxy)ethyl (meth)acrylate, 2-[2-(2-ethoxyethoxy)ethoxy]ethyl (meth)acrylate, 2-methoxyethyl (meth)acrylate, 2-(2-methoxyethoxy)ethyl (meth)acrylate, 2-[2-(2-methoxyethoxy)ethoxy]ethyl (meth)acrylate, hexyl (meth)acrylate, 2-ethylhexyl (meth)acrylate, decyl (meth)acrylate, isodecyl (meth)acrylate, dodecyl (meth)acrylate, 2-hydroxy-3-(prop-2-enoyloxy)propyl 2-methyl-2-propylhexanoate and (5-ethyl-1,3-dioxan-5-yl)methyl (meth)acrylate.

10. The dental, free-radical-polymerizable composition of claim 1, wherein the difunctional (meth)acryloyl compound (A3) is selected from the group consisting of ethylene glycol di(meth)acrylate, diethylene glycol di(meth)acrylate, triethylene glycol di(meth)acrylate, tetraethylene glycol di(meth)acrylate, polyethylene glycol di(meth)acrylate, propylene glycol di(meth)acrylate, dipropylene glycol di(meth)acrylate, tripropylene glycol di(meth)acrylate, tetrapropylene glycol di(meth)acrylate, polypropylene glycol di(meth)acrylate, butane-1,4-diol di(meth)acrylate, butane-1,3-diol di(meth)acrylate, hexane-1,6-diol di(meth)acrylate, decane-1,10-diol di(meth)acrylate, dodecane-1,12-diol di(meth)acrylate, 2-hydroxypropyl 1,3-di(meth)acrylate, 3-hydroxypropyl 1,2-di(meth)acrylate, neopentyl glycol di(meth)acrylate, 2,2-bis[4-[3-(meth)acryloyloxy-2-hydroxypropoxy]phenyl]propane, 2,2-bis[4-(meth)acryloyloxyphenyl]propane, 2,2-bis[4-(meth)acryloyloxyethoxyphenyl]propane, 2,2-bis[4-(meth)acryloyloxydiethoxyphenyl]propane, 2,2-bis[4-(meth)acryloyloxytriethoxyphenyl]propane 2,2-bis[4-(meth)acryloyloxytetraethoxyphenyl]propane, 2,2-bis[4-(meth)acryloyloxypentaethoxyphenyl]propane, 2,2-bis[4-(meth)acryloyloxydipropoxyphenyl]propane, 2,2-bis[4-(meth)acryloyloxyethoxyphenyl]-2-[4-(meth)acryloyloxydiethoxyphenyl]propane, 2-[4-(meth)acryloyloxydiethoxyphenyl]-2-[4-(meth)acryloyloxytriethoxyphenyl]propane, 2-[4-(meth)acryloyloxydipropoxyphenyl]-2-[4-(meth)acryloyloxytriethoxyphenyl]propane, 2,2-bis[4-(meth)acryloyloxyisopropoxyphenyl]propane, ethoxylated bisphenol A di(meth)acrylate, propoxylated bisphenol A di(meth)acrylate, 3(4), 8(9)-bis((meth)acryloyloxymethyl)tricyclo[5.2.1.0^{2,6}]decane, 3,14-dioxa-4,13-dioxo-5,12-diazahexadecane-1,16-diyl di(meth)acrylate, 7,7,9-trimethyl-3,14-dioxa-4,13-dioxo-5,12-diazahexadecane-1,16-diyl di(meth)acrylate, 7,9,9-trimethyl-3,14-dioxa-4,13-dioxo-5,12-diazahexadecane-1,16-diyl di(meth)acrylate, 1,5,5-trimethyl-1-[(2-(meth)acryloyloxyethyl)carbamoylmethyl]-3-(2-(meth)acryloyloxyethyl)carbamoylcyclohexane (CAS 42405-01-6 methacrylate; CAS 42404-50-2 acrylate), 1,1′-[methylenebis(4,1-phenylenimino-carbonyloxy-2,1-ethanediyl)] bis(meth)acrylate (CAS 51243-61-9 methacrylate; CAS 69790-08-5 acrylate) and 1,1′-[methylenebis(2,1-phenyleniminocarbonyloxy-2,1-ethanediyl)] bis(meth)acrylate.

11. The dental, free-radical-polymerizable composition of claim 1, wherein the multifunctional (meth)acryloyl compound (A4) is selected from the group consisting of glycerol tri(meth)acrylate, ethoxylated glycerol tri(meth)acrylate, propoxylated glycerol tri(meth)acrylate, trimethylolethane tri(meth)acrylate, ethoxylated trimethylolethane tri(meth)acrylate, trimethylolpropane tri(meth)acrylate, ethoxylated trimethylolpropane tri(meth)acrylate, ditrimethylolpropane tri(meth)acrylate, ditrimethylolpropane tetra(meth)acrylate, pentaerythritol tri(meth)acrylate, pentaerythritol tetra(meth)acrylate, ethoxylated pentaerythritol tetra(meth)acrylate, dipentaerythritol tri(meth)acrylate, dipentaerythritol tetra(meth)acrylate, dipentaerythritol penta(meth)acrylate, dipentaerythritol hexa(meth)acrylate, ethoxylated dipentaerythritol penta(meth)acrylate, ethoxylated dipentaerythritol hexa(meth)acrylate and (2,4,6-trioxo-1,3,5-triazine-1,3,5(2H,4H,6H)-triyl)tri-2,1-ethanediyl tri(meth)acrylate.

12. The dental, free-radical-polymerizable composition of claim 1, wherein the photoinitiator (B) is selected from the group consisting of 1-hydroxycyclohexylbenzophenone, 4-(2-hydroxyethoxy)phenyl 2-hydroxy-2-propyl ketone, 2-hydroxy-2-methylpropiophenone, 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate (CAS 84434-11-7) and (poly(oxy-1,2-ethanediyl), α,α′,α″-1,2,3-propanetriyltris[ω-[[phenyl(2,4,6-trimethylbenzoyl)phosphinyl]oxy] polymer) (CAS 1834525-17-5), more preferably selected from the group consisting of 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate and (poly(oxy-1,2-ethanediyl),α,α′,α″-1,2,3-propanetriyltris[ω-[[phenyl(2,4,6-trimethylbenzoyl)phosphinyl]oxy] polymer).

13. The dental, free-radical-polymerizable composition of claim 1, wherein
(meth)acryloyl compound (A1) contains at least one urethane group, and/or
(meth)acryloyl compound (A2) does not contain a urethane group, and/or
(meth)acryloyl compound (A3) does not contain a urethane group, and/or
(meth)acryloyl compound (A4) does not contain a urethane group.

14. The dental, free-radical-polymerizable composition of claim 1, for use in stereolithography and/or 3D printing, preferably for the 3D printing of dental shaped components, preferably for the 3D printing of dental transfer splints, IBTs, transfer keys and drilling templates.

15. (canceled)

16. The dental, free-radical-polymerizable composition of claim 1, wherein, in the (meth)acryloyl compound of the formula I, L¹ and/or L² is/are selected from the group consisting of 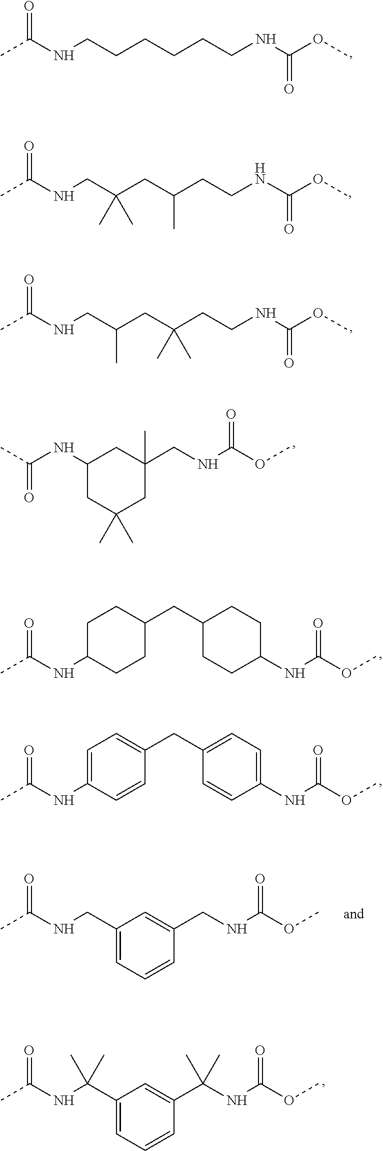
wherein L¹ is attached by the left-hand dashed line to the outer oxygen atom of the polypropylene group (C₃H₆O)ᵥ and is attached by the right-hand dashed line to the outer carbon atom of the polypropylene group (C₃H₆O)_{w}, and/or
wherein L² is attached by the left-hand dashed line to X and is attached by the right-hand dashed line, via an oxygen atom (—O—), to the outer carbon atom of one of the polypropylene groups (C₃H₆O)_{w} or (C₃H₆O)ᵥ.

17. The dental, free-radical-polymerizable composition of claim 16, wherein, in the (meth)acryloyl compound of the formula I, L¹ and/or L² is/are selected from the group consisting of

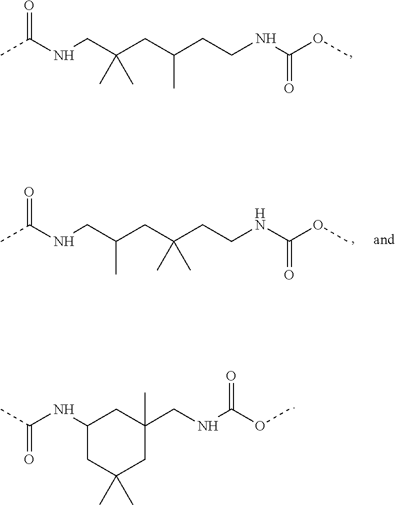

18. The dental, free-radical-polymerizable composition of claim 1, comprising
A1) in an amount of from 40% to 60% by weight,
A2) in an amount of from 20% to 50% by weight,
A3) in an amount of from 2% to 15% by weight,
B) in an amount of from 0.5% to 4% by weight,
in each case based on the total mass of the polymerizable composition.
